(19) United States
(12) Patent Application Publication
(10) Pub. No.: US 2020/0114052 A1
(43) Pub. Date: Apr. 16, 2020

(54) MCS ADVERSE EVENT RISK SCORE

(71) Applicant: HeartWare, Inc., Miami Lakes, FL (US)

(72) Inventors: Michael C. BROWN, Dresher, PA (US); Veronica RAMOS, Homestead, FL (US); Neil VOSKOBOYNIKOV, Miami, FL (US)

(21) Appl. No.: 16/594,552

(22) Filed: Oct. 7, 2019

Related U.S. Application Data
(60) Provisional application No. 62/746,267, filed on Oct. 16, 2018.

(51) Int. Cl. A61M 1/10; F04B 51/00; F04B 15/00; A61M 1/12

(52) U.S. Cl. CPC A61M 1/1086; F04B 51/00; F04B 15/00; A61M 1/122; F04B 2205/09; A61B 5/7275; F04B 2207/70; F04B 2207/01; A61M 2205/3334; A61M 2230/06; F04B 2201/12

(57) ABSTRACT

A method of predicting an adverse event associated with an implantable blood pump including determining a plurality of pump parameters, comparing the plurality of pump parameters to a plurality of threshold values corresponding to the plurality of pump parameters, calculating a weighted sum using the compared plurality of pump parameters to the plurality of threshold values, calculating an adverse event risk score using the calculated weighted sum, and generating an alert when the calculated adverse event risk score deviates from a predetermined value.

### CROSS-REFERENCE TO RELATED APPLICATION

This application claims the benefit of U.S. Application Ser. No. 62/746,267 filed Oct. 16, 2018.

### FIELD

The present technology is generally related to implantable blood pumps.

### BACKGROUND

Mechanical circulatory support devices, such as implantable blood pumps, are used to assist the pumping action of a failing heart. Such blood pumps may include a housing with an inlet, an outlet, and a rotor mounted within the housing. The inlet may be connected to a chamber of a patient's heart, for example the left ventricle, using an inflow cannula. The outlet may be connected to an artery, such as the aorta. Rotation of the rotor drives blood from the inlet towards the outlet and thus assists blood flow from the chamber of the heart into the artery.

Known blood pumps are susceptible to experiencing adverse events which may result in costly hospitalizations and medical interventions for the patient. For example, whether systemic or cardio-pulmonary in nature, adverse events may impact ventricular volume and pressure which is reflected in pump parameters such as power, flow, current, speed, and/or derivatives of pump parameters, such as a patient's circadian cycle, heart rate, aortic valve status, and suction burden. Unfortunately, known systems and methods of detecting adverse events do not provide sufficient advanced predictions of onset and/or fail to consider clinical significance of individual pump parameters and/or derivates thereof.

### SUMMARY

The techniques of this disclosure generally relate to a system and method of calculating an adverse event risk score associated with an implantable blood pump and generating an alert associated therewith.

In one aspect, the present disclosure provides a method of predicting an adverse event associated with an implantable blood pump, the method including determining a plurality of pump parameters; comparing the plurality of pump parameters to a plurality of threshold values corresponding to the plurality of pump parameters; calculating a weighted sum using the compared plurality of pump parameters to the plurality of threshold values; calculating an adverse event risk score using the calculated weighted sum; and generating an alert when the calculated adverse event risk score deviates from a predetermined value.

In another aspect, the method includes determining a plurality of pump operational parameters and using the plurality of pump operational parameters to determine the plurality of pump parameters, the plurality of pump operational parameters including at least one of a group consisting of a power, a flow value, and a pump speed.

In another aspect, the plurality of threshold values includes a power tracking limit and the plurality of pump parameters includes a power deviation with respect to the power tracking limit.

In another aspect, the method includes the plurality of pump parameters includes a suction burden and the plurality of threshold values includes a suction percentage threshold value for comparing to the suction burden.

In another aspect, the method includes the plurality of pump parameters includes a heart rate and the plurality of threshold values includes an arrhythmia value for comparing to the heart rate.

In another aspect, the plurality of pump parameters includes an aortic valve status and the plurality of threshold values includes a threshold open percentage for comparing to the aortic valve status.

In another aspect, the plurality of pump parameters includes a pulsatility level and the plurality of threshold values includes a mean pulsatility level for comparing to the pulsatility level.

In another aspect, the plurality of pump parameters includes a circadian cycle.

In another aspect, the method includes determining a clinical relevance of the plurality of pump parameters with respect to the adverse event and calculating the weighted sum using the clinical relevance.

In another aspect, the method includes assigning a weighted score to the plurality of pump parameters with respect to the clinical relevance.

In another aspect, the predicted adverse event is at least one of a group consisting of a thrombus, a cardiac tamponade, a gastro-intestinal bleeding, a right heart failure, and an arrhythmia.

In one aspect, the disclosure provides a method of calculating an adverse event risk score associated with an implantable blood pump, the method including determining a plurality of pump parameters over a time period; comparing the plurality of pump parameters to a plurality of predetermined values; determining a plurality of weighted scores for each of the compared plurality of pump parameters to the plurality of predetermined values; calculating a weighted sum using the plurality of weighted scores; calculating an adverse event risk score using the calculated weighted sum; and generating an alert when the calculated adverse event risk score deviates from a predetermined value.

In another aspect, the method includes determining a plurality of pump operational parameters and using the plurality of pump operational parameters to determine the plurality of pump parameters.

In another aspect, the method includes determining the plurality of weighted scores according to a clinical relevance with respect to an adverse event.

In another aspect, the plurality of pump parameters includes a power deviation with respect to a power tracking limit.

In another aspect, the plurality of pump parameters includes a suction burden and the plurality of predetermined values includes a suction percentage threshold value for comparing to the suction burden.

In another aspect, the plurality of pump parameters are associated with a cardiac condition of a patient.

In another aspect, the method includes sending the adverse event risk score and the alert to a remote location.

In another aspect, the method includes assigning a severity level to the adverse event risk score.

In one aspect, the disclosure provides a system of predicting an adverse event associated with an implantable blood pump, the system including an implantable blood pump; and a processor in communication with the blood pump, the processor being configured to determine a plurality of pump parameters; compare the plurality of pump parameters to a plurality of threshold values corresponding to the plurality of pump parameters; calculate a weighted sum using the compared plurality of pump parameters to the plurality of threshold values; calculate an adverse event risk score using the calculated weighted sum; and generate an alert when the calculated adverse event risk score deviates from a predetermined value.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a block diagram that illustrates a system including an implantable blood pump and a processor in communication with the blood pump;

FIG. 2 is a flow diagram that illustrates steps associated with a method of determining an adverse event risk score;

FIG. 3 is a flow diagram that illustrates exemplary pump parameters and threshold values used to determine the adverse event risk score;

FIG. 4 depicts five graphs that illustrate windows of log file data produced by the blood pump of FIG. 1;

FIG. 5 is a graph that illustrates a two-week window of log file data produced by the blood pump of FIG. 1, the log file data depicting exemplary pump parameters and exemplary pump operational parameters;

FIG. 6 is a graph that illustrates a two-week window of log file data produced by the blood pump of FIG. 1, the log file data depicting exemplary pump parameters and exemplary pump operational parameters; and

FIG. 7 is a graph that illustrates a two-week window of log file data produced by the blood pump of FIG. 1, the log file data depicting exemplary pump parameters including power deviations with respect to a predetermined value and exemplary pump operational parameters.

### DETAILED DESCRIPTION

Before describing in detail exemplary embodiments, it is noted that the embodiments reside primarily in combinations of device, system components, and processing steps related to calculating an adverse event risk score associated with an implantable blood pump. Accordingly, the device, system, and process components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

As used herein, relational terms, such as “first” and “second,” “top” and “bottom,” and the like, may be used solely to distinguish one entity or element from another entity or element without necessarily requiring or implying any physical or logical relationship or order between such entities or elements. The terminology used herein is for the purpose of describing particular embodiments only and is not intended to be limiting of the concepts described herein. As used herein, the singular forms “a”, “an” and “the” are intended to include the plural forms as well, unless the context clearly indicates otherwise. It will be further understood that the terms “comprises,” “comprising,” “includes” and/or “including” when used herein, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this disclosure belongs. It will be further understood that terms used herein should be interpreted as having a meaning that is consistent with their meaning in the context of this specification and the relevant art and will not be interpreted in an idealized or overly formal sense unless expressly so defined herein.

In embodiments described herein, the joining term, “in communication with” and the like, may be used to indicate electrical or data communication, which may be accomplished by physical contact, induction, electromagnetic radiation, radio signaling, infrared signaling or optical signaling, for example. One having ordinary skill in the art will appreciate that multiple components may interoperate and modifications and variations are possible of achieving the electrical and data communication.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

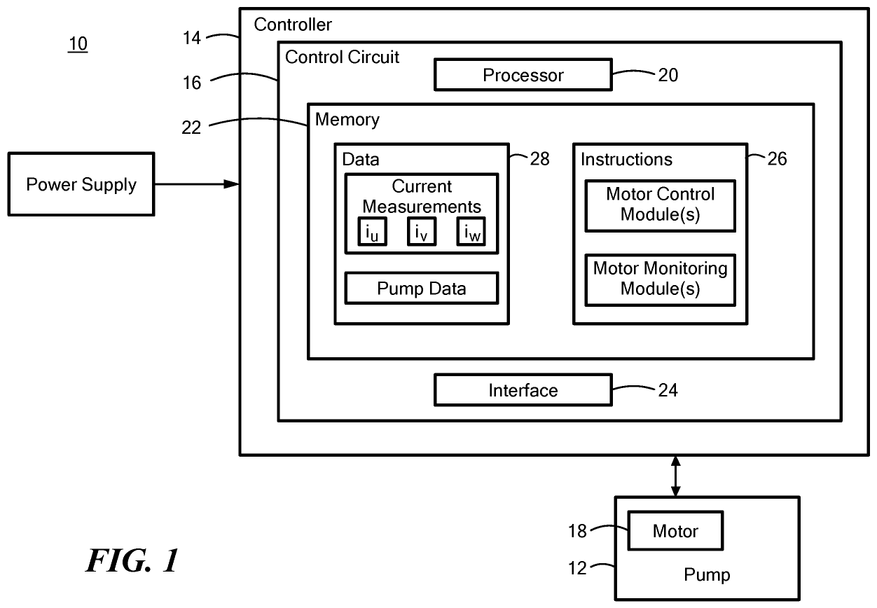

Referring now to the drawings in which like reference designators refer to like elements, there is shown in FIGS. 1-7 an exemplary system for calculating an adverse event risk score constructed in accordance with the principles of the present disclosure and designated generally as “10.” The adverse event risk score is a cumulative weighted score which accounts for a contribution of clinical relevance of blood pump parameters to an adverse event associated with the blood pump. The adverse event risk score is used to predict the adverse event and alert clinicians of the same for corresponding diagnosis, treatment, therapy or the like in an effort to prevent the occurrence of the adverse event. In other words, the adverse event risk score indicates that the patient is at a relatively high risk for the adverse event. The adverse event is an event which poses a health risk to a patient, such as, and without limitation, a thrombus, a cardiac tamponade, a gastro-intestinal bleeding, a right heart failure, an arrhythmia, a stroke, inflow and/or outflow occlusion of a blood pump, or another cardiac event.

FIG. 1 depicts a block diagram of the system 10 including the implantable blood pump 12 in communication with a controller 14. The blood pump 12 may be the HVAD® Pump, the MVAD® Pump, or another mechanical circulatory support device fully or partially implanted within the patient and having a movable element, such as a rotor, configured to pump blood from the heart to the rest of the body. The controller 14 includes a control circuit 16 for monitoring and controlling startup and subsequent operation of a motor 18 implanted within the blood pump 12. The controller 14 may also include a processor 20, a memory 22, and an interface 24 with the memory 22 being configured to store information accessible by the processor 20, including instructions 26 executable by the processor 20 and/or data 28 that may be retrieved, manipulated, and/or stored by the processor 20.

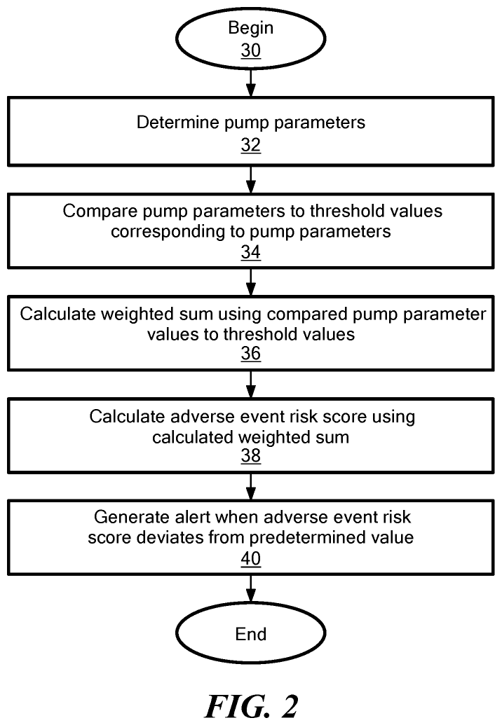

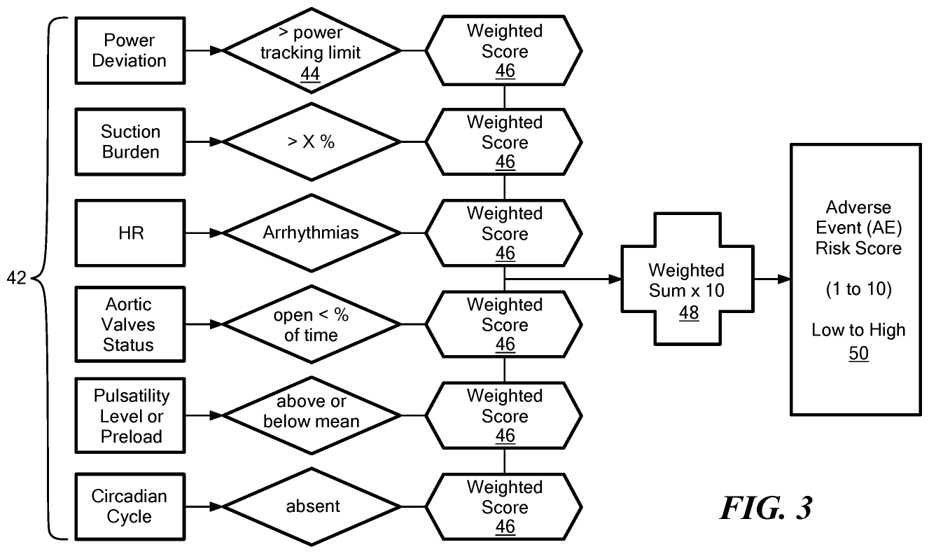

FIG. 2, is a flow chart depicting exemplary method steps used by the system 10 and the processor 20, or another system in communication with the blood pump 12, to calculate the adverse event risk score associated with the blood pump 12. The detailed description of the method steps is provided below with respect to FIGS. 3-7. In one configuration, the method begins at step 30 and proceeds to step 32 including determining one or more pump parameters. The pump parameters may be selectively chosen, such as by a clinician, and a value associated with the pump parameters may be obtained through the pump data 28 (FIG. 1), for example in the form of log file data captured over a time duration such as two weeks. In one configuration, the method includes selecting at least three pump parameters, although two may be selected in other configurations. In step 34, the method includes comparing the pump parameters to one or more threshold values corresponding to the pump parameters. In step 36, a weighted sum is calculated using the comparison between the pump parameters and the threshold values. Proceeding to step 38, the method includes calculating an adverse event risk score using the calculated weighted sum and in step 40, an alert is generated when the calculated adverse event risk score deviates from a predetermined value.

With reference to FIG. 3, a flow diagram depicts exemplary pump parameters 42 compared to corresponding threshold values 44 in a similar or same class or category. The pump parameters 42 are associated with a cardiac condition of a patient. The comparisons between the pump parameters 42 and the corresponding threshold values 44 are each expressed as a weighted score 46, where the weight is assigned to the pump parameters 42 according to the clinical relevance with respect to the adverse event. In other words, the individual pump parameters 42 are assigned a weight based on clinical relevance. The weighted score 46 may be credit or percentage based.

FIG. 3 depicts the pump parameter 42 as a power deviation compared to the threshold value 44 as a power tracking limit. As such, when the pump power deviates from the power tracking limit within the select duration as evidenced by the data, the instances may be recorded and used to determine the weighted score. In the same or other configurations, the pump parameters 42 may include one or more combinations of a suction burden, a heart rate, an aortic valve status, a pulsatility level, and a circadian cycle with the threshold values 44 including a suction percentage threshold value, an arrhythmia value, a threshold open percentage, a mean pulsatility level, and a presence or absence of a circadian cycle, respectively. The list provided herein is exemplary and not intended to be limiting.

The weighted scores 46 are added to determine the weighted sum 48 and thereafter multiplied by a multiplier of 10, or as otherwise selected, to determine the adverse event risk score 50 which is assigned a severity level. FIG. 3 depicts the adverse event risk score 50 as a scale between one to ten with one indicating a low risk and ten indicating a high risk for an adverse event, although other types of scales may be used to indicate the severity of the risk score 50.

The alert is generated when the calculated adverse event risk score 50 deviates from a predetermined value that is a baseline or threshold value determined to indicate a prediction or onset of the adverse event. The predetermined value may be stored in the memory 22 of the processor 20. For example, using the one to ten scale, the predetermined value may be a six on the scale with the alert generated when the adverse event risk score 50 is equal to or greater than six. The alert may be a visual or audible alert shown on a display screen or speaker of the system 10 or transmitted to a remote location for review by a clinician to diagnose specific events and apply appropriate medical therapy or treatment. In another example, the alert and/or the adverse event risk score 50 appear on a processed report, such as an Autolog report. The adverse event risk score 50 itself is communicated with the alert. Depending on the risk score 50 and the following triage of the patient, the risk score 50 may be associated with a particular action plan. For example, a risk score of 7-9 may trigger an alert indicating that the patient immediate proceed to the hospital, whereas an alert of 4-6 may trigger tan alert indicating that the patient make an appointment with a clinician as soon as possible.

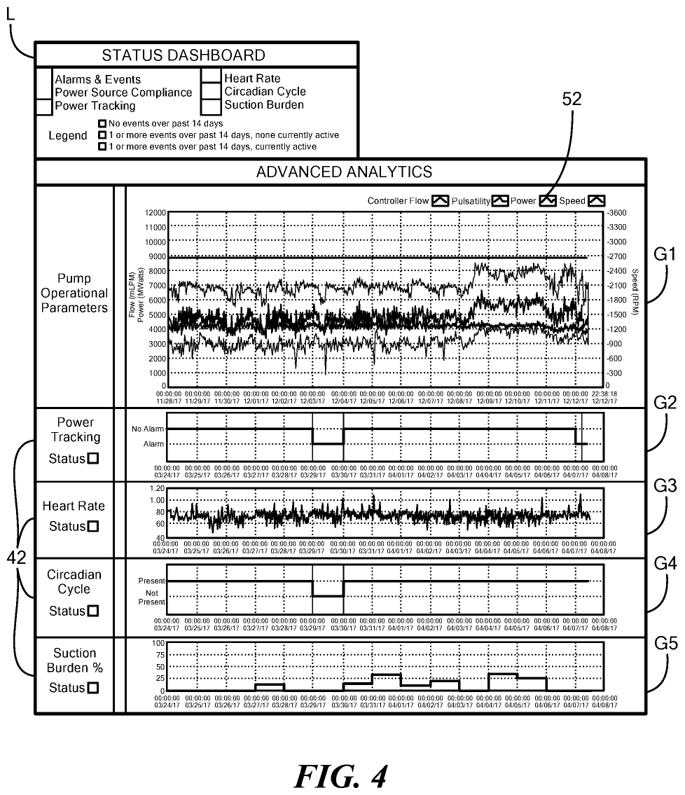

FIG. 4 depicts five graphs reflecting log file data produced by the blood pump 12 and a legend “L” which indicates a presence or absence of an adverse event. The pump parameters 42 are derived from, as shown in graph “G1”, one or more pump operational parameters 52 illustrated as the blood pump's power, flow value, pump speed, and pulsatility as deviations in the pump operational parameters 52 with respect to the threshold values 44 are used to predict the adverse event. As such, the pump operational parameters 52 are used as inputs which provide a summarized output of an overall adverse event. For example, an increase in pump power with respect to a threshold value indicates an onset or presence of thrombus, whereas a relatively low flow condition indicates a suction event. Graphs “G2” through “G5” depict various pump parameters 42.

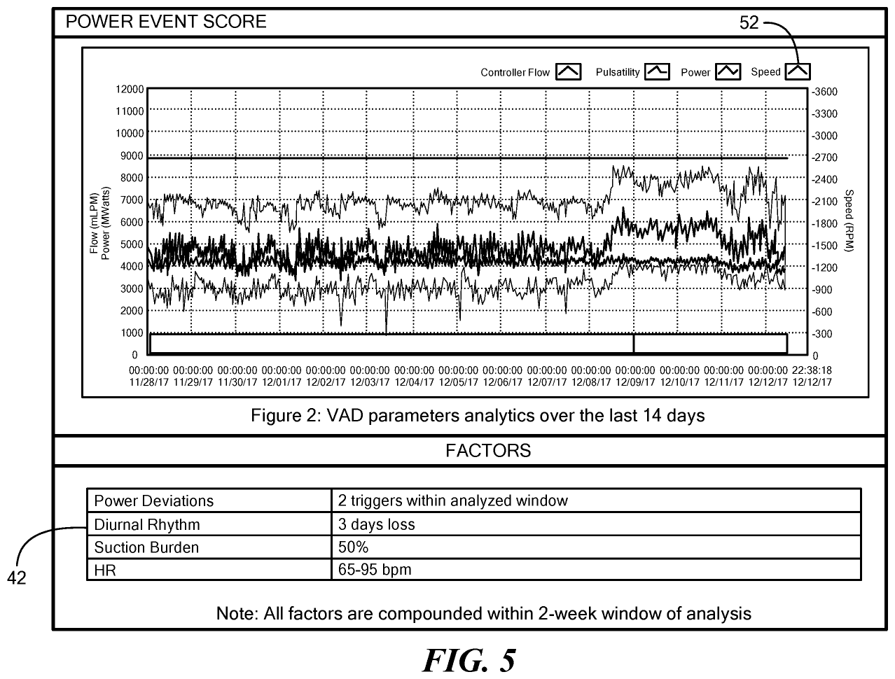

FIG. 5 is a graph illustrating a two-week window of log file data produced by the blood pump 12 including the pump operational parameters 52 plotted within the graph and the pump parameters 42 shown as factors.

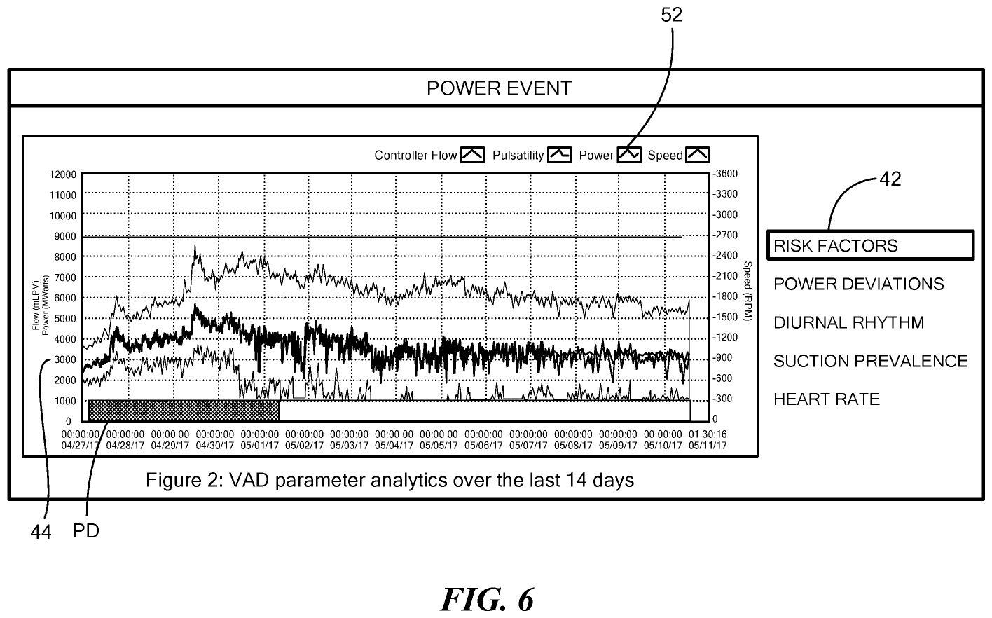

FIG. 6 a graph illustrating a two-week window of log file data produced by the blood pump 12 including the pump power deviating with respect to a threshold value 44 as indicated within a region designated “PD”. The pump parameters 42 indicating risk factors include diurnal rhythm, suction prevalence, and heart rate.

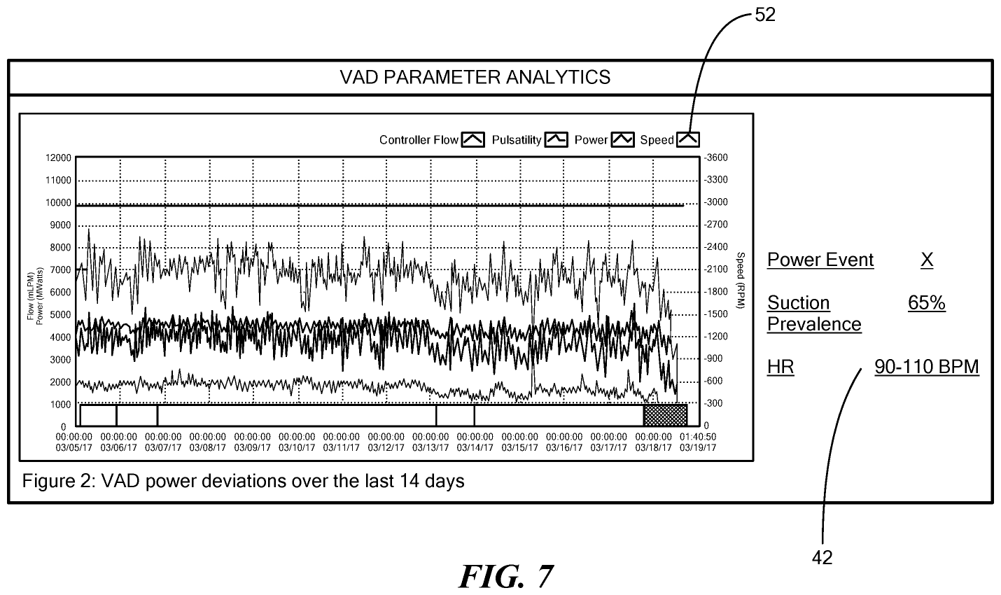

FIG. 7 depicts a graph illustrating a two-week window of log file data produced by the blood pump 12 including the pump operational parameters 52 indicating power deviations and the pump parameters 42 being the adverse event risk factors of power events, suction prevalence, and heart rate.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A method of predicting an adverse event associated with an implantable blood pump, the method comprising:
determining a plurality of pump parameters;
comparing the plurality of pump parameters to a plurality of threshold values corresponding to the plurality of pump parameters;
calculating a weighted sum using the compared plurality of pump parameters to the plurality of threshold values;
calculating an adverse event risk score using the calculated weighted sum; and
generating an alert when the calculated adverse event risk score deviates from a predetermined value.

2. The method according to claim 1, further comprising determining a plurality of pump operational parameters and using the plurality of pump operational parameters to determine the plurality of pump parameters, the plurality of pump operational parameters including at least one of a group consisting of a power, a flow value, and a pump speed.

3. The method according to claim 1, wherein the plurality of threshold values includes a power tracking limit and the plurality of pump parameters includes a power deviation with respect to the power tracking limit.

4. The method according to claim 1, wherein the plurality of pump parameters includes a suction burden and the plurality of threshold values includes a suction percentage threshold value for comparing to the suction burden.

5. The method according to claim 1, wherein the plurality of pump parameters includes a heart rate and the plurality of threshold values includes an arrhythmia value for comparing to the heart rate.

6. The method according to claim 1, wherein the plurality of pump parameters includes an aortic valve status and the plurality of threshold values includes a threshold open percentage for comparing to the aortic valve status.

7. The method according to claim 1, wherein the plurality of pump parameters includes a pulsatility level and the plurality of threshold values includes a mean pulsatility level for comparing to the pulsatility level.

8. The method according to claim 1, wherein the plurality of pump parameters includes a circadian cycle.

9. The method according to claim 1, further comprising determining a clinical relevance of the plurality of pump parameters with respect to the adverse event and calculating the weighted sum using the clinical relevance.

10. The method according to claim 9, further comprising assigning a weighted score to the plurality of pump parameters with respect to the clinical relevance.

11. The method according to claim 1, wherein the predicted adverse event is at least one of a group consisting of a thrombus, a cardiac tamponade, a gastro-intestinal bleeding, a right heart failure, and an arrhythmia.

12. A method of calculating an adverse event risk score associated with an implantable blood pump, the method comprising:
determining a plurality of pump parameters over a time period;
comparing the plurality of pump parameters to a plurality of predetermined values;
determining a plurality of weighted scores for each of the compared plurality of pump parameters to the plurality of predetermined values;
calculating a weighted sum using the plurality of weighted scores;
calculating an adverse event risk score using the calculated weighted sum; and
generating an alert when the calculated adverse event risk score deviates from a predetermined value.

13. The method according to claim 12, further comprising determining a plurality of pump operational parameters and using the plurality of pump operational parameters to determine the plurality of pump parameters.

14. The method according to claim 13, further comprising determining the plurality of weighted scores according to a clinical relevance with respect to an adverse event.

15. The method according to claim 12, wherein the plurality of pump parameters includes a power deviation with respect to a power tracking limit.

16. The method according to claim 12, wherein the plurality of pump parameters includes a suction burden and the plurality of predetermined values includes a suction percentage threshold value for comparing to the suction burden.

17. The method according to claim 12, wherein the plurality of pump parameters are associated with a cardiac condition of a patient.

18. The method according to claim 12, further comprising sending the adverse event risk score and the alert to a remote location.

19. The method according to claim 12, further comprising assigning a severity level to the adverse event risk score.

20. A system of predicting an adverse event associated with an implantable blood pump, the system comprising:
an implantable blood pump; and
a processor in communication with the blood pump, the processor being configured to:
determine a plurality of pump parameters;
compare the plurality of pump parameters to a plurality of threshold values corresponding to the plurality of pump parameters;
calculate a weighted sum using the compared plurality of pump parameters to the plurality of threshold values;
calculate an adverse event risk score using the calculated weighted sum; and
generate an alert when the calculated adverse event risk score deviates from a predetermined value.
